(19) United States
(12) Patent Application Publication
(10) Pub. No.: US 2004/0204487 A1
(43) Pub. Date: Oct. 14, 2004

(54) Novel medicinal product

(76) Inventors: Jean-Pierre Salaun, Landeda (FR); Emmanuelle Plee-Gautier, Brest (FR); Francois Berthou, Brest (FR); Jean-Claude Yvin, Saint Malo (FR)

Correspondence Address: JOHN S. PRATT, ESQ, KILPATRICK STOCKTON, LLP, 1100 PEACHTREE STREET, ATLANTA, GA, 30309, US

(21) Appl. No.: 10/630,374

(22) Filed: Jul. 30, 2003

(30) Foreign Application Priority Data
Aug. 2, 2002 (FR) 02 09911

(51) Int. Cl. A61K 31/202

(52) U.S. Cl. 514/560

(57) ABSTRACT

A medicinal product, which comprises, as active substance, an effective amount of at least one of the metabolites of the group comprising 12-HETE and 11,12-EET.

The invention relates to a novel medicinal product.

It is also directed toward a method for preparing it and its use in combating the differentiation of fibroblasts to adipocytes, from which the fatty layer in the human or animal body is formed.

It is known that the arachidonic acid oxidation cascade in mammals results in metabolites, including in particular

15-deoxy-Δ^{12,14}-prostaglandin J2, or PGJ2, under the action of cyclooxygenase,

12-hydroxyeicosatetraenoic acid, or 12-HETE, under the action of lipoxygenase, and

11,12-epoxyeicosatrienoic acid, or 11,12-EET, under the action of cytochrome P450 of the CYP2C family.

12-Hydroxyeicosatetraenoic acid exists in the form of two stereoisomers, namely 12(S)-HETE and 12(R)-HETE.

The action of 12-lipoxygenase provides the major form, namely 12(S)-HETE.

The stereoisomer 12(R)-HETE can be obtained by reduction of 11,12-EET.

It is also known that PGJ2 is a ligand for a nuclear receptor of the Peroxisome Proliferator Activated Receptor (P.P.A.R.) family. PGJ2 controls the differentiation of pre-adipocyte fibroblasts, i.e. the conversion of these cells to adipocytes. This PGJ2 prostaglandin accelerates the differentiation in question at concentrations of 10⁻⁵ to 10⁻⁶ M.

Now, the accumulation of adipocytes leads to the formation of the fatty layer between the epidermis and the underlying structures.

It is recalled, in this regard, that the differentiation of fibroblasts to adipocytes results in the conversion of the former, which are slender cells lacking in lipid globules, to very rounded cells, the adipocytes, which accumulate large amounts of lipids, in particular of triglycerides, in globules, the mass of which can reach 90% of the cell volume.

This phenomenon can be observed under the microscope.

Now, it is to the applicant company's credit to have found that, among the many metabolites produced by the arachidonic acid oxidative cascade, 12-HETE and 11,12-EET, identified above, are capable of inhibiting the differentiation of fibroblasts to adipocytes.

These two metabolites therefore constitute medicinal products in the sense that they restore, correct or modify organic functions in humans.

Thus, a subject of the invention is, consequently, a medicinal product, which comprises, as active substance, an effective amount of at least one of the metabolites of the group comprising 12-HETE and 11,12-EET.

A subject of the invention is more particularly a medicinal product capable of inhibiting the differentiation of fibroblasts to adipocytes, which comprises, as active substance, an effective amount of at least one of the acids 12-HETE and 11,12-EET.

The term “effective amount” designates an amount of at least one of the acids 12-HETE and 11,12-EET which is at least equal to that with which it is capable of restoring, correcting or modifying at least one organic function in humans or in animals, and in particular of inhibiting the differentiation of fibroblasts to adipocytes.

The invention, by virtue of the inhibition of the differentiation of fibroblasts to adipocytes using the metabolites 12-HETE and 11,12-EET, makes it possible to combat the indiscriminate development of the fatty layers to which reference was made above and, consequently, to combat the establishment in the organism of certain forms of obesity.

The metabolites 12-HETE and 11,12-EET are commercially available.

They are in fact marketed by the company Cayman Chemical (1180 E. Ellsworth Road, Ann Arbor, Mich. 48108 USA) under the trademarks 12(R)-HETE, 12(S)-HETE and (±)11(12)-EpEtrE.

It is also possible to extract them from the red alga Chondrus Crispus using the method which is the subject of the patent application filed by the Applicant company on the same date as the present application, under the title “Method for preparing free polyunsaturated fatty acids and their oxidation metabolites”.

This method consists essentially in reacting, on the alga, a molecule which stimulates its natural defenses so as to cause the production, inter alia, of 12-HETE and of 11,12-EET, and then in extracting them, after approximately 6 to 12 hours, using a chemical extraction method applied to the substrate consisting of the alga.

The property of inhibiting the differentiation of fibroblasts to adipocytes which is exhibited by the metabolites 12-HETE and 11,12-EET was demonstrated by the in vitro assays described below.

For these in vitro assays, the murine adipocyte lines 3T3-F442A and BCF 115 were used.

In addition, cells originating from human fat tissues subsequent to surgical interventions requiring their removal were used. The removed tissues are collected on a bed of ice and immediately treated in the laboratory. The cell culture consists of a mixture of pre-adipocyte cells and adipocyte cells already differentiated.

The pre-adipocyte lines 3T3-F442A (commercial line) and BFC 115 (from Claude Forest INSERM, St. Pères Paris V) are cultured until confluency in Dulbecco's modified Eagle medium (DMEM) (Gibco) in the presence of 5% of newborn calf serum and 5% of fetal calf serum (Gibco).

From confluency, the cells are cultured in a differentiating medium (DMEM with 10% of fetal calf serum and 20 nM of insulin) in the presence or absence of the metabolites to be tested.

The medium is renewed every two or three days.

The differentiation of the cells is evaluated either by morphological criteria (very rounded cells having accumulated large lipid vacuoles) or by measuring the average amount of triglycerides per cell (ratio of the amount of triglyceride per dish to the amount of protein per dish). Under standard culture conditions, in 8 days, 90% of the pre-adipocytes have differentiated to adipocytes.

To show by direct morphological examination the differentiation of the fibroblasts to adipocytes in the presence or absence of the metabolites 12-HETE and/or 11,12-EET, the following procedure was carried out.

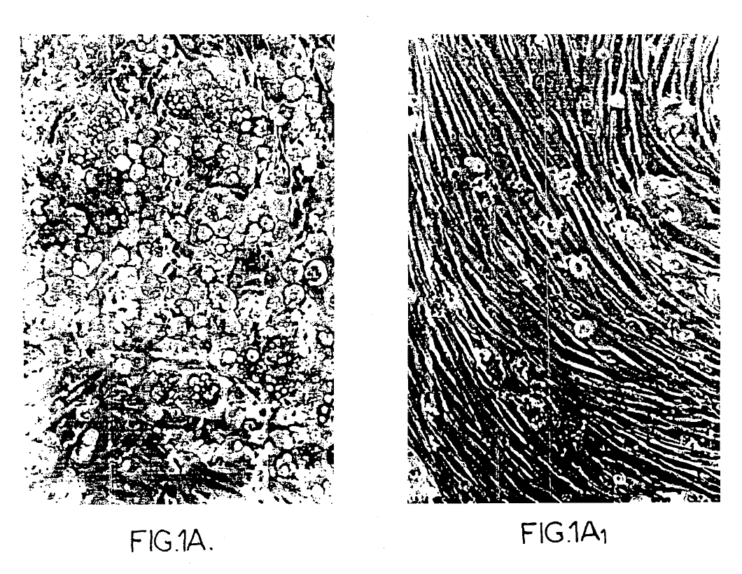

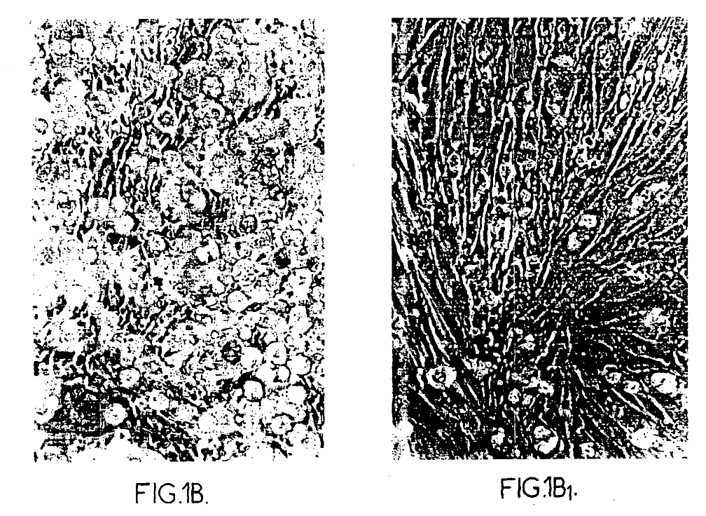

The 3T3-F442A cells are treated, from confluency, with these metabolites. The treatment was renewed every 2 or 3 days throughout the entire differentiation process. The level of differentiation of the cells was evaluated by observation under a photon microscope with a 200 times magnification (see the photos shown in figures FIG. 1A, FIG. 1A₁, FIG. 1B and FIG. 1B₁).

The photo represented in FIG. 1A shows cells which were not placed in the presence of the metabolite used in accordance with the invention, whereas that represented in FIG. 1A₁ shows the cells which were treated at confluency with 10 μM of 12(R)-HETE.

The photo represented in FIG. 1B shows the cells which were treated at confluency with 100 μM of arachidonic acid and that represented in FIG. 1B₁ the cells treated at confluency with 100 μM of arachidonic acid and with 10 μM of 12 (R)-HETE.

Examination of FIGS. 1A and 1B shows great differentiation in the untreated cells (the lipid vacuoles appear refringent in white, FIG. 1A).

Arachidonic acid increases the amount of cells differentiated into adipocytes (FIG. 1B).

When the differentiation process takes place in the presence of 10 μM of 12(R)-HETE, then, 7 days later, the cells appear to be spindle-shaped with very few lipid globules (FIG. 1A₁), even in the presence of arachidonic acid (FIG. 1B₁) which promotes differentiation.

This appearance of the cells is characteristic of undifferentiated cells.

The cells are washed with PBS phosphate buffer (Sigma), recovered from the bottom of the Petri dish by scraping with a spatula, fragmented by ultrasound (2 seconds) and then centrifuged at 10000 g for 20 minutes.

The centrifugation supernatant is removed for triacylglyceride (TAG) analysis.

To determine the average amount of triglycerides per cell at the end of the abovementioned experiments, an automated enzymatic colorimetric test (Roche method) which is based on the studies of Wahlefeld (Wahlefeld and Bergmeyer (eds). Methods of Enzymatic Analysis. 2 second edition. New York Academic Press Inc. 1974: 1831) was used as assaying method.

The 3T3-F442A pre-adipocyte cells were treated from confluency with 12(R)-HETE (10 μM), 12(S)-HETE (10 μM), 11,12-EET (10 μM) and with Δ 15-deoxy prostaglandin J2 (10 μM, Δ 15-deoxyPGJ2) in the presence or absence of 10 μM of 12(R)-HETE. The cell differentiation was evaluated after 7 days by measuring, as indicated above, the amount of triacylglycerides accumulated per mg of protein.

The data thus obtained, given in table I, illustrate the effects of the (S) and (R) stereoisomers of 12-HETE, of 11,12-EET and of 15-deoxyPGJ2 on the differentiation of cells of the 3T3-F442A line.

TABLE I

| | Triacylglycerides |
|---|---|
| Treatment of 3T3-F442A cells | (μg/mg protein) |
| Control (no treatment) | 0.16 |
| 12(R)-HETE (10 μM) | 0.04 |
| 12(S)-HETE (10 μM) | 0.07 |
| 11,12-EET (10 μM) | 0.08 |
| 15-deoxyPGJ2 (10 μM) | 0.52 |
| 15-deoxyPGJ2 (10 μM) and | 0.32 |
| 12(R)-HETE (10 μM) | |

On examining the data given in table I, it appears

that the two (S) and (R) stereoisomers of 12-HETE, and 11,12-EET, at a concentration of 10 μM, greatly inhibit the cell differentiation,

that the ability of 12(R)-HETE to inhibit the differentiation is greater than that of 12(S)-HETE for identical concentrations, and

that 10 μM of 12(R)-HETE inhibit by approximately 40% the differentiation induced by Δ 15-deoxyprostaglandin J2 (PGJ2), the most potent natural inducer of adipocyte differentiation.

The influence of the concentration of metabolites used in accordance with the invention, in the solutions used to inhibit the differentiation of fibroblasts to adipocytes, was also studied.

To do this, 3T3-F442A fibroblasts, or pre-adipocyte cells, were treated from confluency with various concentrations of 12(R)-HETE. The cell differentiation was evaluated after 7 days by measuring the amount of triacylglycerides (TAGs) accumulated per mg of protein (prot).

The concentrations used were 5 μM, 10 μM and 30 μM.

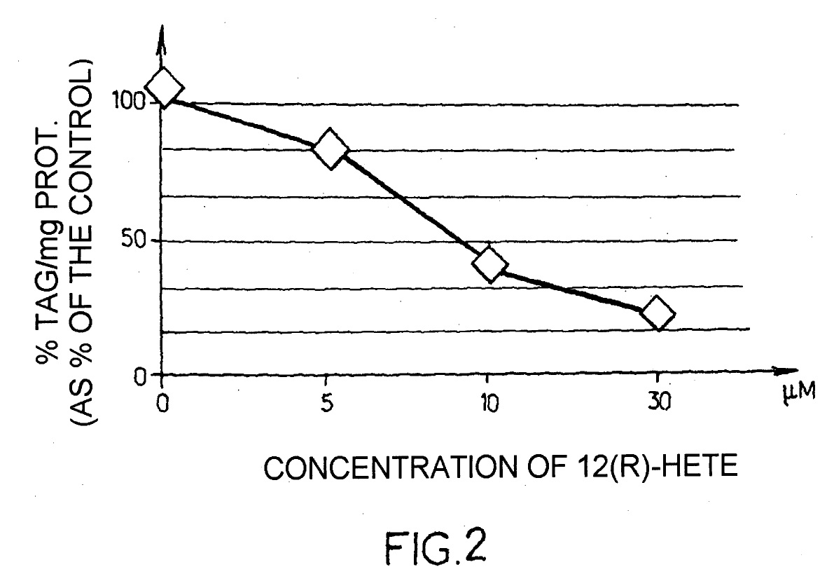

The graph in FIG. 2 represents the variation, in % relative to the control, of the concentration of TAGs (triacylglycerides) accumulated per mg of cellular protein as a function of the concentration of the 12(R)-HETE-based solutions used.

Examination of FIG. 2 makes it possible to note that, when the concentration of 12(R)-HETE increases from 5 μM to 30 μM, the concentration of TAGs in the adipocytes, expressed as % relative to the control (control alone: 100%), decreases from approximately 85% to approximately 25%.

It was also shown that the property of inhibiting the differentiation of fibroblasts to adipocytes was clearly dependent, surprisingly and unexpectedly, on the two metabolites selected in accordance with the invention.

To do this, pre-adipocyte cells of the BFC-115 line (murine line independent of 3T3 F442A) were treated, at a concentration of 10 μM, from confluency with the metabolites selected according to the invention

12(R)-HETE

12(S)-HETE

11,12-EET

and with arachidonic acid, which promotes differentiation.

After 7 days, the average amount of triacylglycerides accumulated per cell was measured, and the following results, given in table II, were obtained, these results showing the effects of the (S) and (R) stereoisomers of 12-HETE, of 11,12-EET and of arachidonic acid on the differentiation of cells of the BFC-115 line.

The BFC-115 pre-adipocyte cells were treated from confluency with 12(R)-HETE (10 μM), 12(S)-HETE (10 μM), 11,12-EET (10 μM) and with arachidonic acid AA (100 μM). The cell differentiation was evaluated after 7 days by measuring the amount of triacylglycerides accumulated per mg of protein.

TABLE II

| Metabolite or fatty acid | Amount of TAG in μg/mg of |
|---|---|
| used | protein |
| Control | =0.45 |
| 12(R)-HETE (10 μM) | =0.17 |
| 12(S)-HETE (10 μM) | =0.28 |
| 11,12-EET (10 μM) | =0.22 |
| AA (100 μM) | =0.55 |

On examining the values given in table II, it is noted that:

the (R) and (S) stereoisomers of 12-HETE, and 11,12-EET, at a concentration of 10 μM, inhibit by approximately 40% the differentiation of the pre-adipocytes of the murine line BFC115,

arachidonic acid clearly promotes the differentiation (FIG. 1B).

It was also determined that the residual level of 12(R)-HETE, present at a concentration of 10 μM in the culture medium of the differentiated 3T3-F442A cells, is respectively 85% and 2.3% after 6 h and 18 h of incubation.

The residual amounts of 12(R)-HETE were analyzed by LC-MS.

The conclusions which may be drawn from this situation are that the metabolite clearly penetrates into the cells.

Three experiments with cells from human adipose tissue show that the metabolites selected in accordance with the invention, used at a concentration of 10 μM, inhibit by 50% the accumulation of triglycerides, measured in a culture of cells grown in pro-differentiating medium and compared to a culture treated with 12(R)-HETE and 11,12-EET.

The possible toxicity of the metabolites selected in accordance with the invention was also studied.

For this purpose, several cellular toxicity markers were measured after 8 days of treatment, namely

the number of cells,

the cell morphology

the LDH (lactate dehydrogenase) activity in the culture medium, a control for membrane permeabilization,

fragmentation of the nucleus by “Hoechst” staining, which is a marker for apoptosis and which is described in Leukemia, 15, 1572-81. Plenchette, S., Moutet M., Benguella M., N'Gondara J. P., Guigner F., Coffe C., Corcos L., Bettaieb A. and Solary E. (2001).

None of these markers responds positively for 12(R)-HETE concentrations of 30 μM.

As regards the dosage and the pharmaceutical forms of administration which can be selected for the medicinal products consisting of or based on the metabolites selected in accordance with the invention, the following will be noted.

By way of examples which are non-limiting but which correspond to advantageous embodiments, indicated below are the compositions of some pharmaceutical formulations, of the type denoted by the expression Over The Counter (OTC) pharmaceutical formulations, dermocosmetic formulations, cosmetic formulations and/or nutraceutical formulations, namely those:

of a cream,

of a gel for local application,

of a gelatin capsule,

of a patch.

1—Composition of a cream in accordance with the invention:

| | |
|---|---|
| Demineralized water | 69.50% |
| Glycerol | 5.00% |
| Acrylate | 0.20% |
| 12-HETE | 0.20% |
| PEG 100 stearate | 4.00% |
| Cetearyl alcohol | 2.00% |
| Preserving agent | 1.00% |
| PEG 40 stearate | 4.00% |
| Vitamin E acetate | 0.50% |
| C12-15 alkyl benzoate | 6.50% |
| Caprylic/capric triglycerides | 5.50% |
| Sodium hydroxide | 1.60% |
| | 100.00% |

2—Composition of a gel for local application and massage intended for the symptomatic treatment of localized excess subcutaneous fat deposits:

Composition per 100 g:

| | |
|---|---|
| 12-HETE | 5 g |

Excipients: fatty acid esters, isopropanol, carboxyvinyl polymer, diethanolamine, deterpeneated natural essences, para-hydroxybenzoate preserving agent, 4-hydroxybenzoic acid methyl ester.

3—Composition of a 20 mg gelatin capsule:

20 mg gelatin capsule composition:

| | |
|---|---|
| 12-HETE | 20 mg |

Excipients: anhydrous lactose, magnesium stearate. Gelatin capsule envelope: gelatin, titanium dioxide (E171), silicon dioxide, sodium lauryl sulfate, indigo carmine (E132).

4—Composition of a dermocosmetic patch intended to give the figure a slimmer appearance:

Composition:

| | |
|---|---|
| Fucus extract | 3.00% |
| 12-HETE | 2.00% |
| Vitamin C palmitate | 1.00% |
| Mixture of polar lipids | 0.15% |
| Vitamin A palmitate | 0.10% |
| Centella asiatica extract | 3.75% |

Film support: polyurethane, release liner: polyester.

Patch 4×3.5 cm in size and 8.8 mm thick, such as, for example, those developed by the company Laboratoires LAVIPHARM, Paris.

The dosage, in particular with respect to the abovementioned pharmaceutical forms, Over The Counter (OTC) pharmaceutical forms, dermopharmaceutical forms, cosmetic forms and/or nutraceutical forms, is advantageously as follows:

in the case of creams, gels and sprays, 2 to 3 concentrated applications on the localized excess subcutaneous fat deposits per day are envisioned, the duration of treatment is approximately 5 weeks.

in the case of gelatin capsules, 1 to 4 gelatin capsules per day are administered for an adult.

in the case of the patches, the patch is applied to the areas concerned every day (or every evening) for at least one month, without stopping the treatment before 1 month.

1-3. Cancelled.

4. A medicinal composition comprising at least one of 12-HETE or 11,12-EET in a form of a cream, a gel, a gelatin capsule or a patch.

5. A method of inhibiting differentiation of a fibroblast to an adipocyte comprising administering to a human or an animal an effective amount of a composition comprising at least one of 12-HETE or 11,12-EET.

6. The method of claim 5, wherein the composition is administered in a form of a cream, a gel, a gelatin capsule or a patch.

7. The method of claim 5, wherein the at least one 12-HETE further comprises 12(R)-HETE.

8. The method of claim 5, wherein the at least one 12-HETE further comprises 12(S)-HETE.

9. A composition comprising a medical product which restores, corrects or modulates an organic function in a human or an animal, comprising an effective amount of 12-HETE, 11, 12-EET or a combination thereof.

10. A method of inhibiting differentiation of a fibroblast to an adipocyte comprising administering to a human or an animal an effective amount of a composition comprising an arachidonic oxidation cascade metabolite, wherein the metabolite is 12-(S)HETE, 12-(R)HETE, 11,12-EET, or a mixture thereof.

11. The method of claim 10, wherein the composition is administered in a form of a cream, a gel, a gelatin capsule or a patch.

12. The method of claim 10, wherein the at least one 12-HETE further comprises 12(R)-HETE.

13. The method of claim 10, wherein the at least one 12-HETE further comprises 12(S)-HETE.

14. A method of treating obesity in an animal or a human comprising administering to the human or the animal an effective amount of a composition comprising an arachidonic acid oxidation cascade metabolite, wherein the metabolite is 12-(S)HETE, 12-(R)HETE, 11,12-EET, or a mixture thereof.

15. The method of claim 14, wherein the composition is administered in a form of a cream, a gel, a gelatin capsule or a patch.

16. The method of claim 14, wherein the at least one 12-HETE further comprises 12(R)-HETE.

17. The method of claim 14, wherein the at least one 12-HETE further comprises 12(S)-HETE.

18. A method of inhibiting an average amount of triacylglycerides per fibroblast or pre-adipocyte cell in an animal or a human comprising administering to the animal or the human an effective amount of a composition comprising 12-HETE, 11,12-EET, or a mixture thereof.
